(19) United States
(12) Patent Application Publication
(10) Pub. No.: US 2026/0335262 A1
(43) Pub. Date: Oct. 29, 2026

(54) METHODS FOR SLOWING HIPPOCAMPAL ATROPHY

(71) Applicant: Alzheon, Inc., Framingham, MA (US)

(72) Inventors: John Hey, Framingham, MA (US); Susan Abushakra, Framingham, MA (US)

(21) Appl. No.: 19/653,246

(22) Filed: Apr. 21, 2026

Related U.S. Application Data
(60) Provisional application No. 63/793,356, filed on Apr. 23, 2025.
(60) Provisional application No. 63/851,155, filed on Jul. 25, 2025.
(60) Provisional application No. 63/891,156, filed on Sep. 30, 2025.

(51) Int. Cl. A61K 31/185; A61P 25/28

(52) U.S. Cl. CPC A61K 31/185; A61P 25/28

(57) ABSTRACT

Provided are methods for preventing, halting, or reversing hippocampal volume atrophy using valiltramiprosate.

### RELATED APPLICATIONS

This application claims the benefit of priority to U.S. Provisional Application No. 63/793,356, filed Apr. 23, 2025, U.S. Provisional Application No. 63/851,155, filed Jul. 25, 2025, and U.S. Provisional Application No. 63/793,356, filed Sep. 30, 2025, the entire contents of each of which are incorporated herein by reference in their entirety.

### BACKGROUND

Reduced hippocampal volume (HV) is an indicator of cognitive decline and is associated with various neurological diseases such as Alzheimer's disease. Reduced HV is one of the earliest markers of AD pathology and precedes overt memory deficits by several years. HV atrophy is most marked in patients with AD pathology versus normal aging or other neurodegenerative diseases. The key role of HV in AD pathogenesis has been extensively documented. For a review, see, S. Abushakra et al., CNS Drugs 2026, 40:199-214.

Valiltramiprosate, also known as ALZ-801, is an investigational drug designed to inhibit the formation of soluble beta amyloid oligomers in the brain. By inhibiting amyloid oligomer formation, valiltramiprosate has the ability to protect neurons from synaptic dystrophy and neuronal loss that manifests as slowing of neurodegeneration and brain atrophy. To this end, valiltramiprosate has been clinically shown to significantly slow HV atrophy, particularly in subjects with mild cognitive impairment (MCI). See e.g., 2025 APOLLOE4 Topline Results Press Release—Alzheon, Inc., Apr. 9, 2025.

### SUMMARY

Applicant has now discovered through phase 2 and phase 3 human clinical trial results, that subjects who have a hippocampal volume at or above a floor threshold and are diagnosed with MCI, rather than mild AD, respond significantly better to treatment with valiltramiprosate. Such subjects demonstrate superior slowing of HV atrophy, delaying or slowing the progression of cognitive decline due to Alzheimer's disease, and slowing of the progression of MCI to mild AD and more severe stages of AD as compared to subjects diagnosed with mild AD and/or subjects having a hippocampal volume below the floor threshold.

For subjects with mild cognitive impairment, slowing of HV atrophy following treatment with valiltramiprosate has now been found to be more pronounced in subjects who have one or more of the following characteristics: 1) an initial HV of at least 6600 mm³, 2) are less than 80 years old (e.g., less than 72 years old, or less than 70 years old), 3) are ApoE4-positive, 4) and have a Mini-Mental State Examination (MMSE) score of 26-30. In some embodiments, the subject must have an initial HV of at least 7000 mm³. In addition, is some instances, atrophy of the hippocampus was reversed over time, i.e., HV increased over baseline volume in early AD subjects termed “super-responders”. The term “hippocampal volume” and the abbreviation “HV” are used herein interchangeably and refer to the bilateral hippocampal volume of a human being.

Therefore, in one aspect, provided are methods for using valiltramiprosate to prevent hippocampal volume atrophy in subjects who have mild cognitive impairment and who have a base hippocampal volume of at least 6600 mm³ and are less than 80 years old prior to treatment. In some embodiments of this aspect, the subjects are less than 72 years old (e.g., less than 70 years old) prior to treatment. In some embodiments of this aspect, the subjects have a base hippocampal volume of at least 7000 mm³.

In another aspect, provided are methods for using valiltramiprosate to reduce the rate of hippocampal volume atrophy in subjects who have mild cognitive impairment and who have a base hippocampal volume of at least 6600 mm³ and are less than 80 years old prior to treatment. In some embodiments of this aspect, the subjects are less than 72 years old (e.g., less than 70 years old) prior to treatment. In some embodiments of this aspect, the subjects have a base hippocampal volume of at least 7000 mm³.

In another aspect, provided are methods for using valiltramiprosate to reduce the rate of age-related hippocampal volume atrophy in subjects (e.g., those who are ApoE4-positive) who are not demonstrating cognitive decline associated with a neurodegenerative disease.

In another aspect, provided are methods for using valiltramiprosate to halt or reverse hippocampal volume atrophy in subjects who have mild cognitive impairment (“MCI”) and who have a base hippocampal volume of at least 6600 mm³ and are less than 80 years old prior to treatment. In some embodiments of this aspect, the subjects are less than 72 years old (e.g., less than 70 years old) prior to treatment. In some embodiments of this aspect, the subjects have a base hippocampal volume of at least 7000 mm³.

In yet another aspect, provided are methods for using valiltramiprosate to delay or slow the progression of cognitive decline only in subjects who are ApoE4-positive and have MCI. In more specific embodiments of this aspect, the subjects to be treated also have a base hippocampal volume of at least 6600 mm³ and are less than 80 years old prior to treatment. In some embodiments of this aspect, the subjects are less than 72 years old (e.g., less than 70 years old) prior to treatment. In some embodiments of this aspect, the subjects have a base hippocampal volume of at least 7000 mm³.

In still another aspect, provides are methods for using valiltramiprosate only in subjects who are ApoE4-positive and have MCI to prevent, delay or slow the transition from MCI to AD. In more specific embodiments of this aspect, the subjects to be treated also have a base hippocampal volume of at least 6600 mm³ and are less than 80 years old prior to treatment. In some embodiments of this aspect, the subjects are less than 72 years old (e.g., less than 70 years old) prior to treatment. In some embodiments of this aspect, the subjects have a base hippocampal volume of at least 7000 mm³.

MCI is generally defined by some memory and/or learning deficits with minimal functional deficits. MCI is also referred to as “Prodromal AD,” particularly in the European Union. The revised criteria for MCI from Alzheimer's Association workgroup (Jack et al. 2024) are as follows. Early MCI is characterized by abnormal serum or cerebral spinal fluid (CSF) biomarkers (pTau and/or Aβ42) and early cognitive deficits. Late MCI is further characterized by more significant cognitive defects and subtle functional deficits. In certain cases, a subject may have an MMSE as low as 24, but will still be characterized as having late MCI, rather than mild AD due to meeting other parameters set forth herein.

Diagnosing MCI and distinguishing it from Mild AD (where functional deficits become more readily apparent, but still remain mild) typically involves one or more cognitive tests alone or in conjunction with one or more biological analyses, such as volumetric MRI measuring hippocampal volume or whole brain volume; Diffusion Tensor Imaging to determine mean diffusivity of extracellular water in white matter or gray matter; Aβb42, p-Tau181, MBTR-Tau, p-Tau231, pTau217, and/or any other clinically predictive pTau isoform biomarker levels in serum or CSF; ratio of p-Tau217/nTaupTau-217 (phosphorylated-Tau217 over non-phosphorylated Tau217), p-Tau217/Abeta42, pTau231/npTau231, pTau231/Ab42, or pTau231/t-Tau; or PET imaging to determine the presence of amyloid and/or Tau in the brain.

In some of the foregoing aspects, a subject is determined to have MCI if they have an MMSE score of 27-30 e.g., from 6-months or less, or no less than 60 days, prior to beginning valiltramiprosate treatment. Alternatively, a subject is determined to have MCI if they meet one or more of the following: (1) one or more different cognitive test thresholds; (2) one or more cognitive test thresholds coupled with one or more brain volumetric thresholds as determined by volumetric MRI; (3) one or more cognitive test thresholds coupled with one or more brain microstructure thresholds as determined by Diffusion Tensor Imaging (DTI); (4) one or more cognitive test thresholds coupled with one or more plasma or cerebral spinal fluid biomarker thresholds; or (5) one or more cognitive test thresholds coupled with brain amyloid and/or Tau thresholds as determined by positron emission tomography (PET), each of which is set forth in detail below.

### BRIEF DESCRIPTION OF THE FIGURES

FIG. 1 illustrates the QSP modeled relationship between hippocampal volume and age (solid lines); and between plasma AB42/40 ratio (dashed lines) as a function of apolipoprotein E (APOE) genotype in subjects that will eventually manifest MCI and AD.

FIG. 2 shows the reduction in hippocampal atrophy in all valiltramiprosate treated ApoE4 homozygous subjects vs placebo.

FIG. 3 shows the reduction in hippocampal atrophy in mild cognitive impairment (MCI, MMSE 27-30) valiltramiprosate treated subjects vs placebo.

FIG. 4 compares the hippocampal atrophy changes over 78 weeks of treatment with valiltramiprosate between the super responder and normal responder patient populations.

FIG. 5 compares the ADAS-Cog13 values over 78 weeks of treatment with valiltramiprosate between the super responder and normal responder patient populations.

FIG. 6 shows the change in HV at 78 weeks vs HV baseline for subjects who have mild cognitive impairment and were treated with valiltramiprosate.

FIG. 7 is a slope analysis that represents the time to disease progression in subject with mild cognitive impairment, showing a significant slowing of slope of decline, derived from the clinical data shown in FIG. 3.

FIG. 8 represents a slope that represents the time to disease progression in subjects with mild Alzheimer's disease, showing non-significant slowing of slope of decline.

FIG. 9 is a graph depicting the effects of 265 mg BID dosing of valiltramiprosate on the change in cognitive Rey Auditory Verbal Learning Test (RAVLT) scores from baseline in human subjects with MCI (MMSE 27-30) compared with human subjects with Mild AD (MMSE 22-26) over a four-year period in a human Phase 2 clinical trial.

FIG. 10 is a graph depicting the effects of 265 mg BID dosing of valiltramiprosate on the change in cognitive Rey Auditory Verbal Learning Test (RAVLT) scores from baseline in ApoE3/4 heterozygote human subjects with MCI (MMSE 27-30) compared with human subjects with Mild AD (MMSE 22-26) over a four-year period in a human Phase 2 clinical trial.

FIG. 11 is a graph depicting the effects of 265 mg BID dosing of valiltramiprosate on the change in Rey Auditory Verbal Learning Test (RAVLT) scores from baseline in ApoE4/4 homozygote human subjects with MCI (MMSE 27-30) compared with human subjects with Mild AD (MMSE 22-26) over a four-year period in a human Phase 2 clinical trial.

FIG. 12 is a graph depicting the effects of 265 mg BID dosing of valiltramiprosate on the change in Clinical Dementia Rating-Sum of Boxes (CDR-SB) scores from baseline in human subjects with MCI (MMSE 27-30) compared with human subjects with Mild AD (MMSE 22-26) over a four-year period in a human Phase 2 clinical trial.

FIG. 13 is a graph depicting the effects of 265 mg BID dosing of valiltramiprosate on the change in Clinical Dementia Rating-Sum of Boxes (CDR-SB) scores from baseline in ApoE3/4 heterozygote human subjects with MCI (MMSE 27-30) compared with human subjects with Mild AD (MMSE 22-26) over a four-year period in a human Phase 2 clinical trial.

FIG. 14 is a graph depicting the effects of 265 mg BID dosing of valiltramiprosate on the change in Clinical Dementia Rating-Sum of Boxes (CDR-SB) scores from baseline in ApoE4/4 homozygote human subjects with MCI (MMSE 27-30) compared with human subjects with Mild AD (MMSE 22-26) over a four-year period in a human Phase 2 clinical trial.

FIG. 15 is a graph depicting the effects of 265 mg BID dosing of valiltramiprosate on the change in Mini-Mental State Examination (MMSE) scores from baseline in human subjects with MCI (MMSE 27-30) compared with human subjects with Mild AD (MMSE 22-26) over a four-year period in a human Phase 2 clinical trial.

FIG. 16 is a graph depicting the effects of 265 mg BID dosing of valiltramiprosate on the change in Mini-Mental State Examination (MMSE) scores from baseline in ApoE3/4 heterozygote human subjects with MCI (MMSE 27-30) compared with human subjects with Mild AD (MMSE 22-26) over a four-year period in a human Phase 2 clinical trial.

FIG. 17 is a graph depicting the effects of 265 mg BID dosing of valiltramiprosate on the change in Mini-Mental State Examination (MMSE) scores from baseline in ApoE4/4 homozygote human subjects with MCI (MMSE 27-30) compared with human subjects with Mild AD (MMSE 22-26) over a four-year period in a human Phase 2 clinical trial.

FIG. 18 is a set of bar graphs depicting the effects of 265 mg BID dosing of valiltramiprosate or a placebo on the decline in ADAS-Cog scores (panel A), the decline in CDR-SB scores (panel B), and the change in hippocampal volume (HV) (panel C) over 78 weeks in a human Phase 3 clinical trial of ApoE4/4 homozygote MCI subjects grouped by their baseline HV (≥6600 mm³ or <6600 mm³). P-values are from a T-test analysis of these results and reflect the difference between valiltramiprosate- and placebo-treated subjects in each subgroup.

FIG. 19 is a set of bar graphs depicting the effects of 265 mg BID dosing of valiltramiprosate or a placebo on the decline in ADAS-Cog scores (panel A), the decline in CDR-SB scores (panel B), and the change in hippocampal volume (HV) (panel C) over 78 weeks in a human Phase 3 clinical trial of ApoE4/4 homozygote MCI subjects grouped by their baseline HV (≥7000 mm³ or <7000 mm³). P-values are from a T-test analysis of these results and reflect the difference between valiltramiprosate- and placebo-treated subjects in each subgroup.

FIG. 20 is a set of bar graphs depicting the effects of 265 mg BID dosing of valiltramiprosate or a placebo on the decline in ADAS-Cog scores (panel A), the decline in CDR-SB scores (panel B), and the change in hippocampal volume (HV) (panel C) over 78 weeks in a human Phase 3 clinical trial of ApoE4/4 homozygote MCI subjects grouped by their baseline HV (≥7200 mm³ or <7200 mm³). P-values are from a T-test analysis of these results and reflect the difference between valiltramiprosate- and placebo-treated subjects in each subgroup.

### DETAILED DESCRIPTION

As part of a first embodiment, provided is a method of preventing hippocampal volume atrophy in a selected subject suffering from mild cognitive impairment comprising the step of administering to the subject an amount of valiltramiprosate that is at least 265 mg BID, wherein the subject is selected only if the subject has a base hippocampal volume of at least 6600 mm³ and is less than 80 years old upon the initial administration of valiltramiprosate. In some parts of the first embodiment, the subject is less than 70 years old upon initial administration of valiltramiprosate. In some embodiments of this aspect, the subjects have a base hippocampal volume of at least 7000 mm³. In some embodiments of this aspect, the subjects have a base hippocampal volume of at least 7200 mm³.

As part of a second embodiment, provided is method of reducing the rate of hippocampal volume atrophy in a selected subject suffering from mild cognitive impairment comprising the step of administering to the subject an amount of valiltramiprosate that is at least 265 mg BID, wherein the subject is selected only if the subject has a base hippocampal volume of at least 6600 mm³ and is less than 80 years old upon the initial administration of valiltramiprosate. In some parts of the second embodiment, the subject is less than 70 years old upon initial administration of valiltramiprosate. In some embodiments of this aspect, the subjects have a base hippocampal volume of at least 7000 mm³. In some embodiments of this aspect, the subjects have a base hippocampal volume of at least 7200 mm³.

As part of a third embodiment, provided is a method of halting or reversing hippocampal volume atrophy in a selected subject suffering from mild cognitive impairment comprising the step of administering to the subject an amount of valiltramiprosate that is at least 265 mg BID, wherein the subject is selected only if the subject has a base hippocampal volume of at least 6600 mm³ and is less than 80 years old upon the initial administration of valiltramiprosate. In some parts of the third embodiment, the subject is less than 70 years old upon initial administration of valiltramiprosate. In some embodiments of this aspect, the subjects have a base hippocampal volume of at least 7000 mm³. In some embodiments of this aspect, the subjects have a base hippocampal volume of at least 7200 mm³.

As part of a fourth embodiment, the subject in the described methods (e.g., as in any one of the first to third embodiments) is selected only if the subject has a base hippocampal volume of at least 7250 mm³, at least 7500 mm³, at least 7750 mm³, at least 8000 mm³, at least 8250 mm³, at least 8500 mm³, at least 8750 mm³, or at least 9000 mm³. Alternatively, the subject in the described methods (e.g., as in any one of the first to third embodiments) is selected only if the subject has a base hippocampal volume of from 7000 mm³ to 9000 mm³, from 7250 mm³ to 9000 mm³, from 7500 mm³ to 9000 mm³, from 7750 mm³ to 9000 mm³, from 8000 mm³ to 9000 mm³, from 8250 mm³ to 9000 mm³, from 8500 mm³ to 9000 mm³, from 7000 mm³ to 8500 mm³, from 7500 mm³ to 8500 mm³, from 7750 mm³ to 8500 mm³, or from 8000 mm³ to 8500 mm³.

As part of a fifth embodiment, the subject in the described methods (e.g., as in any one of the first to fourth embodiments) is only selected if the subject is less than 69 years old, less than 68 years old, less than 67 years old, less than 66 years old, less than 65 years old, less than 64 years old, or less than 63 years old upon the initial administration of valiltramiprosate. In other aspects, the subject in the described methods (e.g., as in any one of the first to fourth embodiments) is only selected if the subject is from 60 years old to 69 years old, from 60 years old to 67 years old, from 60 years old to 65 years old, from 62 years old to 65 years old, or from 63 years old to 65 years old upon the initial administration of valiltramiprosate.

As part of a sixth embodiment, the subject in the described methods (e.g., as in any one of the first to fifth embodiments) is only selected if the subject is ApoE4-positive. In some aspects, the subject in the described methods (e.g., as in any one of the first to fifth embodiments) is only selected if the subject is ApoE4 homozygous.

As part of a seventh embodiment, the subject in the described methods (e.g., as in any one of the first to sixth embodiments) is only selected if the subject has an MMSE score of 24-30. In some aspects, the subject in the described methods (e.g., as in any one of the first to sixth embodiments) is only selected if the subject has an MMSE score of 26-30. In some aspects, the subject in the described methods (e.g., as in any one of the first to sixth embodiments) is only selected if the subject has an MMSE score of 27-30. In some aspects, the subject in the described methods (e.g., as in any one of the first to sixth embodiments) is only selected if the subject has an MMSE score of 24-30 as measured no more than 6 months (e.g., no more than 60 days) prior to the initial administration of valiltramiprosate. In some aspects, the subject in the described methods (e.g., as in any one of the first to sixth embodiments) is only selected if the subject has an MMSE score of 26-30 as measured no more than 6 months (e.g., no more than 60 days) prior to the initial administration of valiltramiprosate. In some aspects, the subject in the described methods (e.g., as in any one of the first to sixth embodiments) is only selected if the subject has an MMSE score of 27-30 as measured no more than 6 months (e.g., no more than 60 days) prior to the initial administration of valiltramiprosate. In some aspects, the subject in the described methods (e.g., as in any one of the first to sixth embodiments) is only selected if the subject has an MMSE score of 24-30 as measured no more than 30 days prior to the initial administration of valiltramiprosate. In some aspects, the subject in the described methods (e.g., as in any one of the first to sixth embodiments) is only selected if the subject has an MMSE score of 26-30 as measured no more than 30 days prior to the initial administration of valiltramiprosate. In some aspects, the subject in the described methods (e.g., as in any one of the first to sixth embodiments) is only selected if the subject has an MMSE score of 27-30 as measured no more than 30 days prior to the initial administration of valiltramiprosate.

As part of an eighth embodiment, provided is a method of reducing the rate of age-related hippocampal volume atrophy in a selected subject comprising the step of administering to the subject an amount of valiltramiprosate that is at least 265 mg BID, wherein the subject is selected only if the subject is not demonstrating any cognitive impairment that is associated with a neurodegenerative disease.

As part of a ninth embodiment, the subject as described in the eighth embodiment is ApoE4-positive. Alternatively, the subject as described in the eighth embodiment is ApoE4 homozygous.

As part of a tenth embodiment, the subject as described in the eighth embodiment or ninth embodiment, is less than 80 years old or less than 70 years old. In some parts of the tenth embodiment, the subject is less than 65 years old, less than 64 years old, less than 63 years old, less than 62 years old, less than 61 years old, less than 60 years old, less than 59 years old, less than 58 years old, less than 57 years old, less than 56 years old, or less than 55 years old.

As part of an eleventh embodiment, provided is a method of reducing the likelihood of a subject suffering from mild-cognitive impairment (MCI) progressing to Alzheimer's disease (AD), comprising administering to the subject an amount of valiltramiprosate that is at least 265 mg BID. The term “reducing the likelihood” as used herein means preventing, delaying, or slowing, Such subjects may be characterized by any one or combination of the features described in e.g., the fourth to seventh embodiments.

As part of a twelfth embodiment, provided is a method of preventing hippocampal atrophy (HV) in subjects suffering from late MCI having an MMSE score of about 24 to about 26.

As part of a thirteenth embodiment, provided is a method of halting or reversing hippocampal atrophy (HV) in subjects suffering from late MCI having an MMSE score of about 24 to about 26.

In some aspects, the subjects with MCI or Mild AD as described herein, particularly subjects with MCI, who are candidates for preventing, halting or reversing HV atrophy are those who are in the 90th or greater percentile of HV (i.e., subjects whose baseline HV is in the 90th or greater percentile) as compared to normal, age-matched subjects (i.e., subjects who do not have MCI or AD). In some aspects, the subjects with MCI or Mild AD as described herein, particularly subjects with MCI, who are candidates for preventing, halting or reversing HV atrophy are those who are under 80 years of age or under 70 years of age at the time treatment begins. In some aspect, the subjects are those who are under 70 years of age, under 65 years of age, or under 60 years of age at the time treatment begins.

As part of a fourteenth embodiment, provided is a method of selecting a subject for treatment with valiltramiprosate only if the subject is (i) ApoE4-positive; and (ii) has MCI (and not mild or more severe degrees of AD); and then treating the selected subject with an effective amount of valiltramiprosate. In some aspects of this embodiment, a subject is determined to have MCI if the subject has an MMSE score of between 27-30. In some aspects of this embodiment, the MMSE score is obtained within sixty (60) days prior to treating the subject.

As part of a fifteenth embodiment, provided is a method of selecting and treating a subject to delay or slow the progression of cognitive decline due to Alzheimer's disease comprising the steps of: a. selecting a subject only if the subject is ApoE4-positive and has mild cognitive impairment as determined by having an MMSE score of between 27 and 30 measured less than sixty days prior to treatment; and b. treating the selected subject with valiltramiprosate. In some aspects of this embodiment, the MMSE score is obtained within sixty (60) days prior to treating the subject. In some parts of this embodiment, the subject is selected only if the subject also has a baseline hippocampal volume of at least of at least 6600 mm³. In some parts of this embodiment, the subject is selected only if the subject also has a baseline hippocampal volume of at least 7000 mm³, at least 7200 mm³, at least 7250 mm³, at least 7500 mm³, at least 7750 mm³, at least 8000 mm³, at least 8250 mm³, at least 8500 mm³, at least 8750 mm³, or at least 9000 mm³. In some parts of this embodiment, the subject is selected only if the subject also is less than 80 years old, In some parts of this embodiment, the subject is selected only if the subject also is less than 70 years old, less than 69 years old, less than 68 years old, less than 67 years old, less than 66 years old, less than 65 years old, less than 64 years old, or less than 63 years old upon the initial administration of valiltramiprosate. In more specific parts of this embodiment, the subject is selected only if the subject also has a baseline hippocampal volume of at least 7000 mm³ and is also less than 80 years old. In other more specific parts of this embodiment, the subject is selected only if the subject also has a baseline hippocampal volume of at least 7000 mm³ and is also less than 70 years old.

As part of a sixteenth embodiment, provided is a method of selecting and treating a subject to delay or slow the progression of cognitive decline due to Alzheimer's disease (e.g., slowing the progression of MCI to mild AD and more severe stages of AD) comprising the steps of: a. selecting a subject only if the subject is ApoE4-positive and has mild cognitive impairment. In some parts of this sixteenth embodiment, the subject is only selected if the subject also has a hippocampal volume of greater than 6600 mm³. In some parts of this sixteenth embodiment, the subject is only selected if the subject also has a hippocampal volume of greater than 7000 mm³. In some parts of this sixteenth embodiment, the subject is only selected if the subject also has a hippocampal volume of greater than 7200 mm³.

In any of the foregoing first through sixteenth embodiments, MCI is distinguished from mild AD and is determined by one or more of the following: (1) one or more different cognitive test thresholds; (2) one or more cognitive test thresholds coupled with one or more brain volumetric thresholds as determined by volumetric MRI; (3) one or more cognitive test thresholds coupled with one or more brain microstructure thresholds as determined by Diffusion Tensor Imaging (DTI); (4) one or more cognitive test thresholds coupled with one or more plasma or cerebral spinal fluid biomarker thresholds; or (5) one or more cognitive test thresholds coupled with brain amyloid and/or Tau thresholds as determined by positron emission tomography (PET).

In some parts of the first through sixteenth embodiment, a subject is determined to have MCI if they have an MMSE score of 24-30, a CDR-G score of 0.5, and a CDR memory box score of >0.5. CDR-G is a global score from the Clinical Dementia Rating tests and is on a scale of 0 to 3. A score of 0.5 means the subject has questionable dementia. The memory box score is a score from the Memory domain subtest of CDR. A score of >0.5 of the memory box portion of the CDR means the subject has at least moderate memory loss that is more pronounced for short-term memory and that such memory loss may interfere with daily activities.

In some parts of the first through sixteenth embodiment, a subject is determined to have MCI if they have a MOCA score of 18-30, a CDR-G score of 0.5, and a CDR memory box score of >0.5. MOCA is the Montreal Cognitive Assessment and is used as an early screening test for cognitive dysfunction. Generally, MOCA scores of 18-25 indicate mild impairment, while scores of 26-30 indicate normal cognitive ability.

In some parts of the first through sixteenth embodiment, a subject is determined to have MCI if they have an MMSE score of 24-30, a CDR-G score of 0.5, a CDR memory box score of >0.5, and a RBANS-DM index of ≤105. RBANS is the Repeatable Battery for the Assessment of Neuropsychological Status. RBANS-DM is the delayed memory portion of RBANS. An RBANS-DM score of 100 is considered average, and scores between 85-115 are considered normal variation from average.

In some parts of the first through sixteenth embodiment, a subject is determined to have MCI if they have a MOCA score of 18-30, a CDR-G score of 0.5, a CDR memory box score of >0.5, and a RBANS-DM index of ≤105.

In some parts of the first through sixteenth embodiment, a subject is determined to have MCI if they have an MMSE score of 24-30, a MOCA score of 18-30, a CDR-G score of 0.5, a CDR memory box score of >0.5, and a Buschke's SRT score of that is more than 1 standard deviation (SD) from the age-adjusted mean. Buschke SRT is the Buschke Selective Reminding Tests. It evaluates complex verbal memory by measuring a subject's ability to recall information under varying conditions.

In some parts of the first through sixteenth embodiment, a subject is determined to have MCI if they have a MOCA score of 18-30, a CDR-G score of 0.5, a CDR memory box score of >0.5, and a Buschke's SRT score that is more than 1 SD from the age-adjusted mean.

In some parts of the first through sixteenth embodiment, a subject is determined to have MCI if they have an MMSE score of 24-30, a CDR-G score of 0.5, a CDR memory box score of >0.5, and an ISLT z-score of ≤−1. ISLT is the Cogstate International Shopping List Test. It is a is a word list learning test that looks at total recall, delayed recall and retention-weighted recall. Z-scores compare the subject's performance on the test with a baseline mean sample. Z-scores below-1 indicate some memory impairment.

In some parts of the first through sixteenth embodiment, a subject is determined to have MCI if they have an MOCA score of 18-30, a CDR-G score of 0.5, a CDR memory box score of >0.5, and an ISLT z-score of ≤−1.

In some parts of the first through sixteenth embodiment, a subject is determined to have MCI if they have an MMSE score of 24-30, a CDR-G score of 0.5, a CDR memory box score of >0.5, and a WMS-LM score less than 1 SD of age-adjusted mean. WMS-LM is the Wechsler Memory Scale-Logical Memory subtest. It measures verbal episodic memory with the LM subtest including LM1 (immediate recall) and LM II (delayed recall) that are used for MCI diagnosis.

In some parts of the first through sixteenth embodiment, a subject is determined to have MCI if they have a MOCA score of 18-30, a CDR-G score of 0.5, a CDR memory box score of >0.5, and a WMS-LM score less than 1 SD of age-adjusted mean.

In some parts of the first through sixteenth embodiment, a subject is determined to have MCI if they have an MMSE score of 24-30, a CDR-G score of 0.5, a CDR memory box score of >0.5 and a CVLT T score that is less than 1 SD of age-adjusted mean (of the most current test version). The CVLT is the California Verbal Learning Test. It measures verbal memory and learning, involving hearing and recalling lists of related words over five trials, including immediate recall, delayed recall (after a nonverbal task), and recognition.

In some parts of the first through sixteenth embodiment, a subject is determined to have MCI if they have a MOCA score of 18-30, a CDR-G score of 0.5, a CDR memory box score of >0.5, and a CVLT T score that is less than 1 SD of age-adjusted mean.

In some parts of the first through sixteenth embodiment, a subject is determined to have MCI if they have an MMSE score of 24-30, a CDR-G score of 0.5, a CDR memory box score of >0.5, and a RBANS-DM index of ≤105.

In some parts of the first through sixteenth embodiment, a subject is determined to have MCI if they have a MOCA score of 18-30, a CDR-G score of 0.5, a CDR memory box score of >0.5, and a RBANS-DM index of ≤105.

In some parts of the first through sixteenth embodiment, a subject is determined to have MCI if they have an MMSE score of 24-30, a CDR-G score of 0.5, a CDR memory box score of >0.5, a RBANS-DM index of ≤105, and either a mean diffusivity in the hippocampus of ≤2.01 mm²/sec or a mean diffusivity in the medial temporal cortex of ≤0.93 mm²/sec as measured by DTI. DTI is Diffusion Tensor Imaging. It is an advanced MRI technique that measures microstructural integrity by tracking the diffusion of water molecules in the brain. The larger the mean water diffusivity, the more free the water movement indicating injury and loss of brain tissue, whereas lower mean diffusivity indicates preserved microstructural complexity and tissue integrity.

In some parts of the first through sixteenth embodiment, a subject is determined to have MCI if they have a MOCA score of 18-30, a CDR-G score of 0.5, a CDR memory box score of >0.5, a RBANS-DM index of ≤105, and either a mean diffusivity in the hippocampus of ≤2.01 mm²/sec or a mean diffusivity in the medial temporal cortex of ≤0.93 mm²/sec as measured by DTI.

In some parts of the first through sixteenth embodiment, a subject is determined to have MCI if they have an MMSE score of 24-30, a CDR-G score of 0.5, a CDR memory box score of >0.5, a RBANS-DM index of ≤105, and either a mean diffusivity in the fornix of ≤1.21 mm²/sec or a mean diffusivity in the genu of the corpus callosum of ≤0.86 mm²/sec as measured by DTI.

In some parts of the first through sixteenth embodiment, a subject is determined to have MCI if they have a MOCA score of 18-30, a CDR-G score of 0.5, a CDR memory box score of >0.5, a RBANS-DM index of ≤105, and either a mean diffusivity in the fornix of ≤1.21 mm²/sec or a mean diffusivity in the genu of the corpus callosum of ≤0.86 mm²/sec as measured by DTI.

In some parts of the first through sixteenth embodiment, a subject is determined to have MCI if they have an MMSE score of 24-30, a CDR-G score of 0.5, a CDR memory box score of >0.5, a RBANS-DM index of ≤105, and a plasma pTau217/Aβ42 ratio above the threshold of the assay. PTau217 refers to a form of Tau protein that is phosphorylated at amino acid 217. Elevated plasma levels of pTau217 are correlated with amyloid buildup in the brain. Aβ42 is a form of amyloid-beta. Lower plasma levels of Aβ can indicate the beginnings of plaque formation in the brain. Thus, a higher plasma pTau217/Aβ42 ratio indicates amyloid buildup in the brain. Various assays can be used to measure a pTau217/Aβ42 ratio and depending upon the assay used, the threshold will be different. One example of such an assays is Fujirebio Diagnostic's Lumipulse® assay. The Lumipulse® G pTau217/β-Amyloid 1-42 Plasma Ratio is an in vitro test using human plasma that combines the results of Lumipulse® G pTau217 Plasma and Lumipulse® G β-Amyloid 1-42-N Plasma assays into a ratio of pTau217 to β-Amyloid 1-42 concentrations using the LUMIPULSE® G1200 System. A test result≤0.00370 is a negative result (below the threshold), which is consistent with patients who are unlikely to have amyloid pathology. A test result of ≥0.00738 is a positive result (above the threshold), which is consistent with patients who are likely to have amyloid pathology. A test result between 0.00371 and 0.00737 is an indeterminate result (but also below the threshold), which is consistent with patients who are uncertain to have amyloid pathology. ApoE4 homozygotes who are positive for amyloid by PET or pTau217 CSF levels, tend to show lower plasma pTau217 than corresponding ApoE4 heterozygotes. Therefore, in some embodiments, a pTau217/Aβ42 ratio above 0.00371 is considered positive for ApoE4 homozygotes.

In some parts of the first through sixteenth embodiment, a subject is determined to have MCI if they have an MOCA score of 24-30, a CDR-G score of 0.5, a CDR memory box score of >0.5, a RBANS-DM index of ≤105, and a plasma pTau217/Aβ42 ratio above the threshold of the assay (i.e., ≥0.00738 using the Lumipulse® G pTau217 Plasma and Lumipulse® G β-Amyloid 1-42-N Plasma assays).

In some parts of the first through sixteenth embodiment, a subject is determined to have MCI if they have a MMSE score of 24-30, a CDR-G score of 0.5, a CDR memory box score of >0.5, a RBANS-DM index of ≤105, and a plasma or CSF pTau217 level above the positivity threshold of the assay. In both the Lumipulse® G pTau217 Plasma assay and the Simoa® ALZpath pTau217 assay, a plasma level of >0.274 pg/ml is generally considered a positive result and therefore above the threshold. However, some studies use a two cutoff system where plasma levels below 0.22 pg/ml is considered negative, levels above 0.34 pg/ml are considered positive and levels between 0.22 and 0.34 pg/ml are considered uncertain. Thus, in some embodiments a pTau217 plasma level above 0.22 pg/ml is considered above the positivity threshold. In still other embodiments, a pTau217 plasma level above 0.20 pg/ml is considered above the positivity threshold. In some embodiments, a pTau217 plasma level above 0.15 pg/ml is considered above the positivity threshold for ApoE4 homozygotes due to the fact that these subjects who are positive for amyloid by PET or pTau217 CSF levels, tend to show lower plasma pTau217 than corresponding ApoE4 heterozygotes, sometimes below any of the 0.274 pg/ml, 0.22 pg/ml, or 0.20 pg/ml cutoffs set forth above. For pTau217 CSF levels using the Simoa® ALZpath pTau217 assay values above 10.45 pg/ml or above 11.9 pg/ml are considered a positive result and therefore above the threshold.

In some parts of the first through sixteenth embodiment, a subject is determined to have MCI if they have a MMSE score of 26-30, a CDR-G score of 0.5, a RBANS-DM index of ≤95, and positivity for plasma pTau217.

In some parts of the first through sixteenth embodiment, a subject is determined to have MCI if they have a MMSE score of 26-30, a CDR-G score of 0.5, a RBANS-DM index of ≤105, and positivity for plasma pTau217.

In some parts of the first through sixteenth embodiment, a subject is determined to have MCI if they have a MMSE score of 26-30, a CDR-G score of 0.5, a RBANS-DM index of ≤95, and positivity for plasma pTau217/Aβ42 ratio.

In some parts of the first through sixteenth embodiment, a subject is determined to have MCI if they have a MMSE score of 26-30, a CDR-G score of 0.5, a RBANS-DM index of ≤105, and positivity for plasma pTau217/Aβ42 ratio.

In some parts of the first through sixteenth embodiment, a subject is determined to have MCI if they have a MOCA score of 18-30, a CDR-G score of 0.5, a CDR memory box score of >0.5, a RBANS-DM index of ≤105, and either a plasma pTau217 level above the positivity threshold, or a plasma Aβ42 below the threshold of the assay (indicating more aggregated Aβ42 in the brain).

In some parts of the first through sixteenth embodiment, a subject is determined to have MCI if they have a MMSE score of 24-30, a CDR-G score of 0.5, a CDR memory box score of >0.5, a RBANS-DM index of ≤105, and a plasma or CSF Tau-MTBR level above the positivity threshold of the assay. Tau-MTBR is the microtubule binding region of Tau. Most Tau-MTBR assays measure a species called MTBR-Tau243, a fragment of the Tau protein containing amino acid 243. See K Horie et al., 2025, Nature Medicine, 31, 2044-2053 (2025). There is no single, universally established cutoff level for plasma or CSF MTBR-Tau243 levels in clinical practice and therefore the determination of a positivity threshold will depend on the assay and will be made by the laboratory testing the sample.

In some parts of the first through sixteenth embodiment, a subject is determined to have MCI if they have a MOCA score of 18-30, a CDR-G score of 0.5, a CDR memory box score of >0.5, a RBANS-DM index of ≤105, and a plasma or CSF Tau-MTBR level above the threshold of the assay.

In some parts of the first through sixteenth embodiment, a subject is determined to have MCI if they have a MMSE score of 24-30, a CDR-G score of 0.5, a CDR memory box score of >0.5, a RBANS-DM index of ≤105, and a plasma or CSF pTau181 level above the positivity threshold of the assay. PTau181 refers to a form of Tau protein that is phosphorylated at amino acid 181. Elevated plasma levels of pTau181 are correlated with amyloid buildup in the brain. Aβ42 is a form of amyloid-beta. In the Quanterix's LucentAD pTau181 test, plasma levels of ≥14.2 pg/ml are considered positive (i.e., over the threshold). Another test for pTau181 levels is Quanterix's Simoa® pTau-181 Advantage PLUS test where the cutoff for positivity is >16.5 pg/ml.

In some parts of the first through sixteenth embodiment, a subject is determined to have MCI if they have a MOCA score of 18-30, a CDR-G score of 0.5, a CDR memory box score of >0.5, a RBANS-DM index of ≤105, and a plasma or CSF pTau181 level above the positivity threshold of the assay.

In some parts of the first through sixteenth embodiment, a subject is determined to have MCI if they have a MMSE score of 24-30, a CDR-G score of 0.5, a CDR memory box score of >0.5, a RBANS-DM index of ≤105, and a centiloid value of >24-30 in an amyloid PET scan. Centiloids are a standardized unit for measuring amyloid in the brain using an amyloid PET scan. The centiloid scale ranges from 0 to 100 with higher values indicating more amyloid.

In some parts of the first through sixteenth embodiment, a subject is determined to have MCI if they have a MOCA score of 18-30, a CDR-G score of 0.5, a CDR memory box score of >0.5, a RBANS-DM index of ≤105, and a centiloid value of >24-30 in an amyloid PET scan.

In some parts of the first through sixteenth embodiment, a subject is determined to have MCI if they have a MMSE score of 24-30, a CDR-G score of 0.5, a CDR memory box score of >0.5, a RBANS-DM index of ≤105, and a Tau-SUVR score between 1.1 and 1.46 in a subject's medial and lateral temporal lobe. Tau-SUVR, which stands for Tau-Standardized Uptake Value Ratio, is a quantitative measure used in Tau-PET imaging in the brain. SUVR divides the radioactive signal (tracer uptake) in a target brain region (e.g., temporal lobe) by the signal in a reference region (typically the cerebellum, which is not affected in MCI).

In some parts of the first through sixteenth embodiment, a subject is determined to have MCI if they have a MOCA score of 18-30, a CDR-G score of 0.5, a CDR memory box score of >0.5, a RBANS-DM index of ≤105, and a Tau-SUVR score between 1.1 and 1.46 in a subject's medial and lateral temporal lobe.

It will be understood by those of skill in the art that the cutoff values for specific biomarkers (pTau217, pTau181, MTBR-Tau243, and Aβ42) are typically set by the laboratory testing the subject's plasma or CSF samples. Therefore, the cutoff values set forth above are only intended to be a general guideline. The determination of whether or not a subject has a pTau217 plasma or CSF level above a threshold (or a pTau217/Aβ42 ratio above a threshold) and therefore is considered positive for that biomarker will be made by the particular laboratory running the assay.

In each of the foregoing embodiments, subjects who are not selected by the various parameters set forth are not treated with valiltramiprosate. In other words, only selected subjects are to be treated with valiltramiprosate.

As used herein, the term “subject” or “patient” are synonymous and refer to a human.

Valiltramiprosate may be administered orally or intravenously. In one aspect, administration is oral. In some aspects of any of the foregoing embodiments, Valiltramiprosate is dosed at 265 mg BID. In other aspects of any of the foregoing embodiments, Valiltramiprosate is dosed at 265 mg TID and even more frequently.

Unless otherwise stated, hippocampal volume atrophy refers to bilateral hippocampal volume loss.

### EXEMPLIFICATION

### Example 1. Phase 3 Human Clinical Trial Results

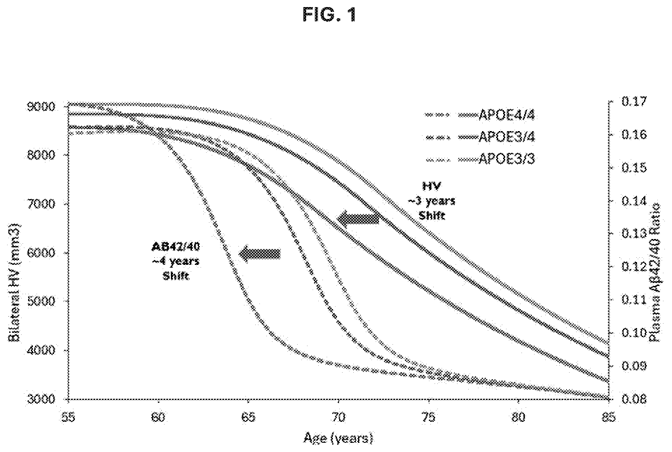

Valiltramiprosate was investigated in a 78-week double-blind, placebo-controlled, two-arm Phase 3 human clinical trial that randomized 325 ApoE4 homozygotes to placebo or 265 mg BID valiltramiprosate, and stratified by mild cognitive impairment (MCI, MMSE 27-30) or Mild AD (MMSE 22-26). It was previously established that the rate of hippocampal volume decline correlates to the severity and/or rate of cognitive decline. See e.g., WO 2020/257159. As shown by FIG. 1, Quantitative Systems Pharmacology (QSP) modeling establishes that untreated subjects who are ApoE4 homozygous (APOE4/4) have a significantly earlier onset of, and more drastic, HV decline than APOE3/3 and APOE3/4 genotypes. Coupled with a similar, substantial shift in plasma Aβ42/Aβ40 ratio, ApoE4 homozygotes represent a distinct form of subjects to treat since they demonstrate an earlier onset of symptoms leading to accelerated disease progression.

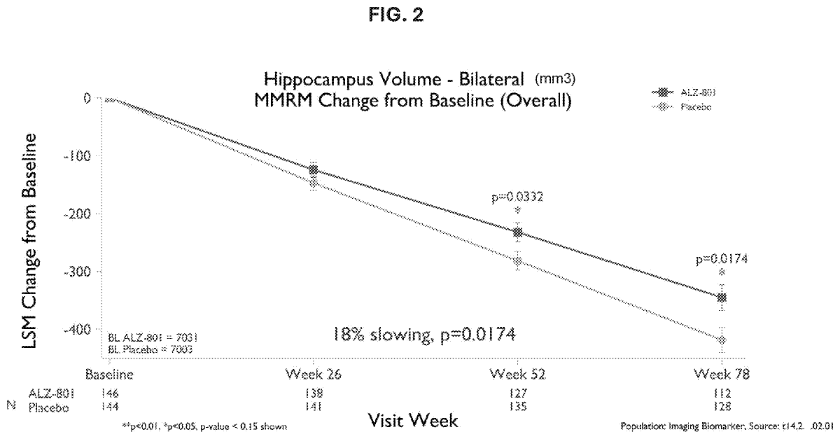

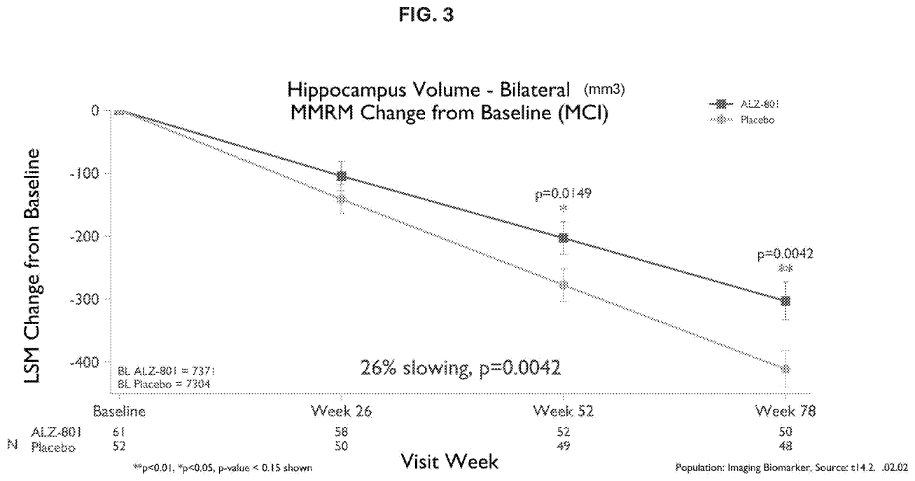

The primary outcome of the trial was ADAS-Cog13 (Alzheimer's Disease Assessment Scale-Cognitive Subscale) and secondary outcomes included CDR-SB (Clinical Dementia Rating-Sum of Boxes), DAD (Disability Assessment for Dementia), and hippocampal volume (HV). Overall, valiltramiprosate consistently reduced atrophy in hippocampus and all other brain regions, indicating substantive neuroprotective benefits. For example, FIG. 2 shows that valiltramiprosate slowed bilateral HV atrophy by 18% (Δ=+74 mm³; p=0.0174) in the overall population (APOE4 homozygotes; MMSE 22-30), and by 26% (4=+108 mm³; p=0.0042) in the MCI population (APOE4 homozygotes; MMSE 27-30) (FIG. 3). HV changes in MCI subjects were negatively correlated to ADAS-Cog13 (r=−0.339; p=0.02) and CDR-SB (r=−0.414; p<0.01) and positively correlated to DAD (r=0.569; p<0.001). This effect and correlation was also consistently seen across all other vMRI measures, including cortical thickness, cortical thickness Mayo index in medial temporal lobe, ventricular volume and whole brain volume. For ADAS and CDR, negative correlations with HV mean, better clinical function correlates with less HV atrophy. For DAD, positive correlations with HV mean, better functional scores correlate with less HV atrophy.

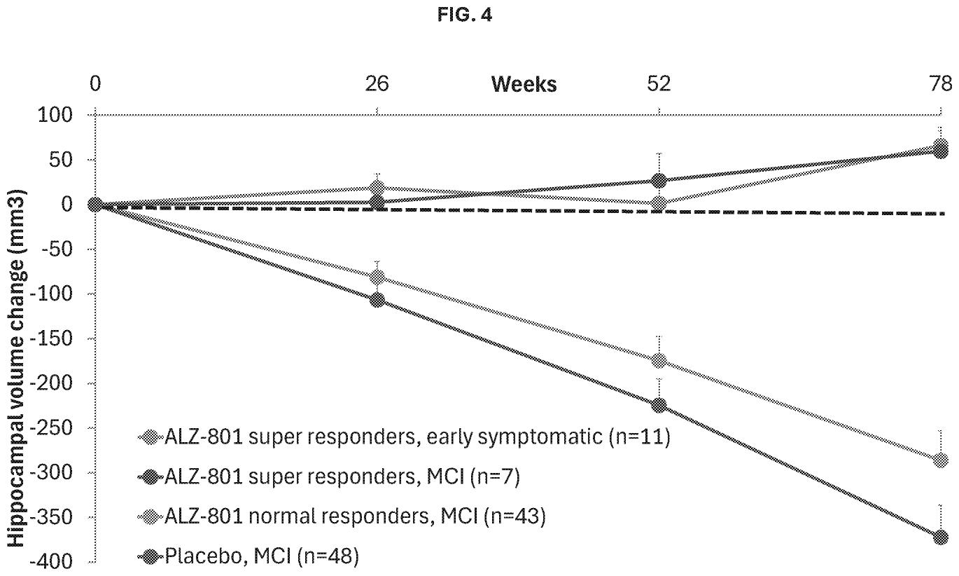

Interestingly, in a subset of subjects from the MCI group, treatment with valiltramiprosate not only halted HV atrophy, but in some cases, HV atrophy was reversed. See the “super responders” shown in FIG. 4. Super responder was defined as an observed increase of HV at 78 weeks over baseline. The characteristics of the subset of subjects represented in FIG. 4 are provided in the table below.

| | | | Screening | |
|---|---|---|---|---|
| | | MMSE | MMSE | Baseline HV |
| Group | Age | (range) | (average) | (mm³) |
| Super responder, early | 63.7 ± 2.4 | 30-24 | 27.7 ± 0.6 | 7755.1 ± 418.7 |
| symptomatic | | | | |
| Super responder, MCI | 63.6 ± 3.3 | 30-27 | 29.0 ± 0.4 | 8085.7 ± 524.5 |
| Normal responder, | 67.4 ± 0.9 | 30-27 | 28.4 ± 0.2 | 7286.3 ± 122.3 |
| MCI | | | | |
| Placebo, MCI | 67.7 ± 0.9 | 30-27 | 28.1 ± 0.1 | 7323.6 ± 127.8 |

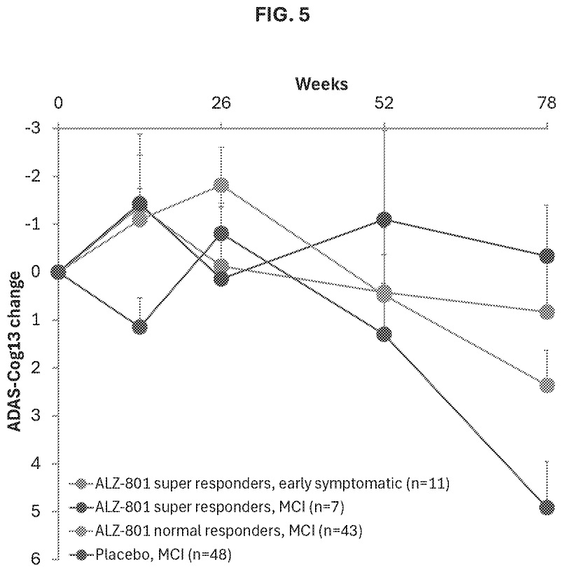

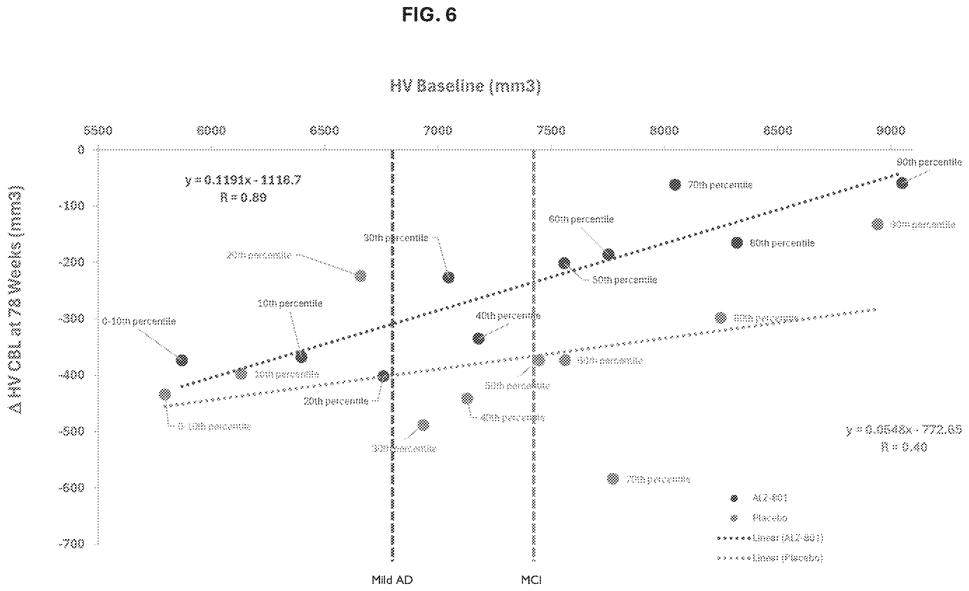

As shown above, within the MCI population, subjects who were between the age of about 60 years old to about 67 years old with an MMSE range of 30-27 and a baseline HV of between about 7500 and about 8700, showed prevention of hippocampal atrophy over 78 weeks, and, remarkably, an increase in HV in certain instances. Further, as shown by FIG. 5, there was a greater stabilization of ADAS-Cog13 values in MCI super responders when compared to normal responders following treatment. FIG. 6 shows that MCI subjects with a higher baseline HV had better protection against HV atrophy when treated with valiltramiprosate than those with lower baseline HV. This data establishes a trend that MCI subjects with higher HV values have a decreased disease progression burden.

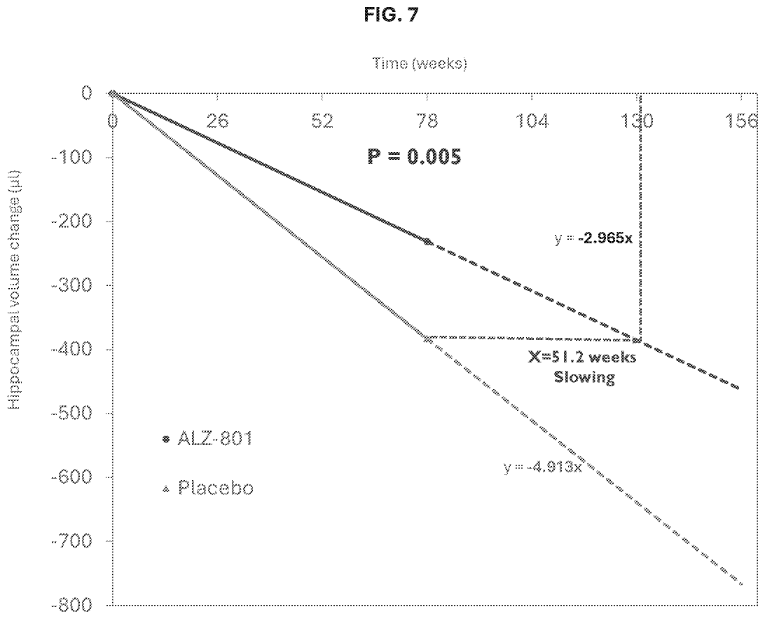

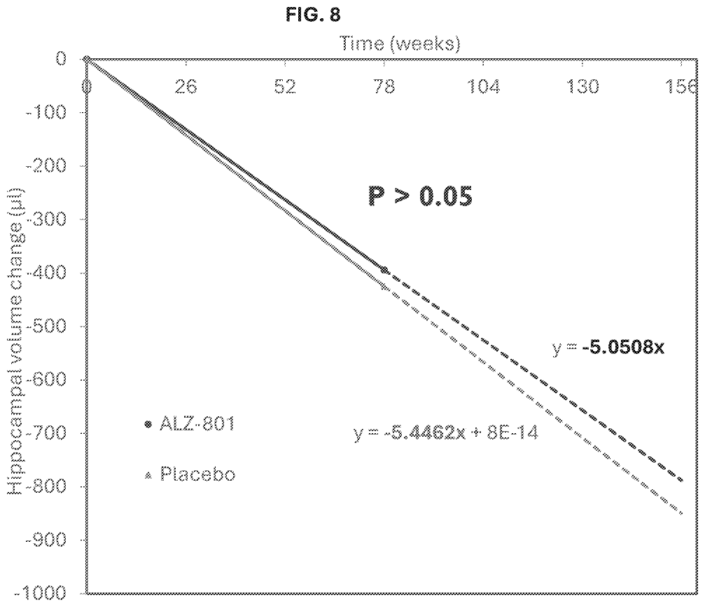

FIG. 7 further establishes that, as a whole, MCI subjects who are treated with valiltramiprosate have approximately a 1 year delay in disease progression whereas subjects with mild Alzheimer's disease have essentially no change over placebo (FIG. 8).

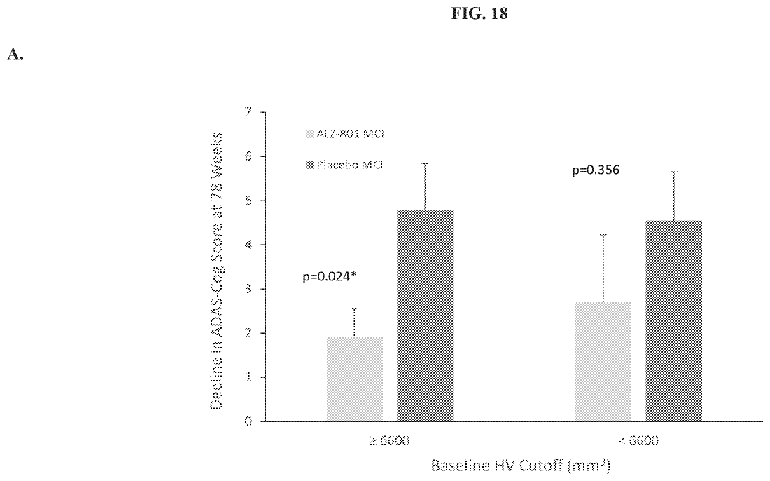

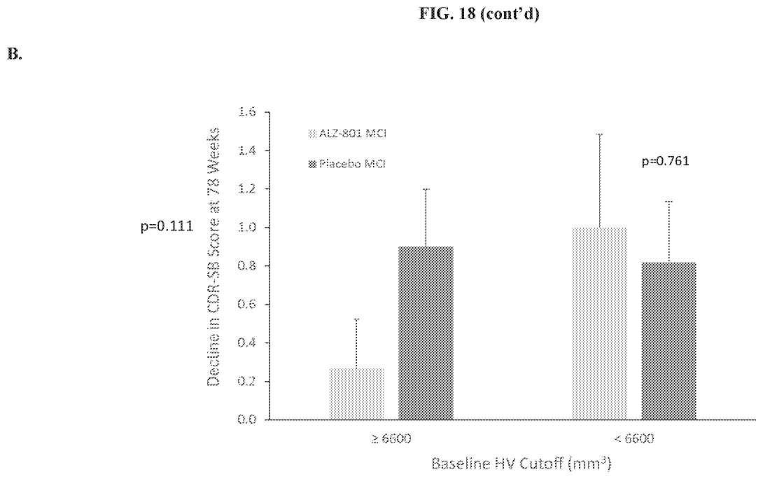

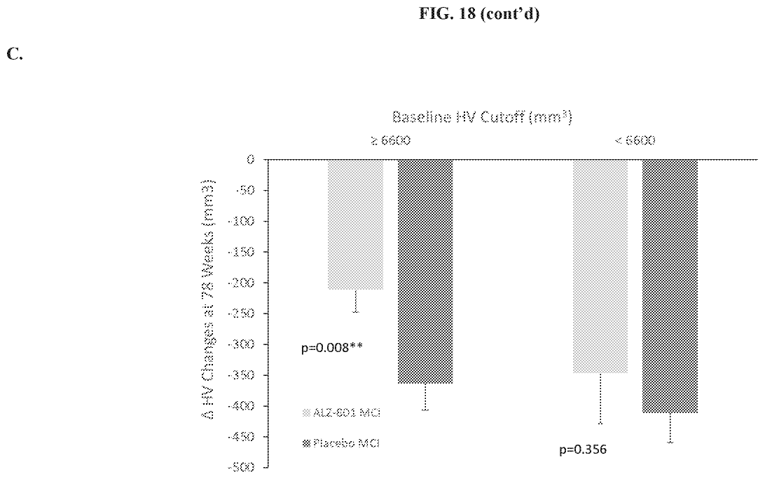

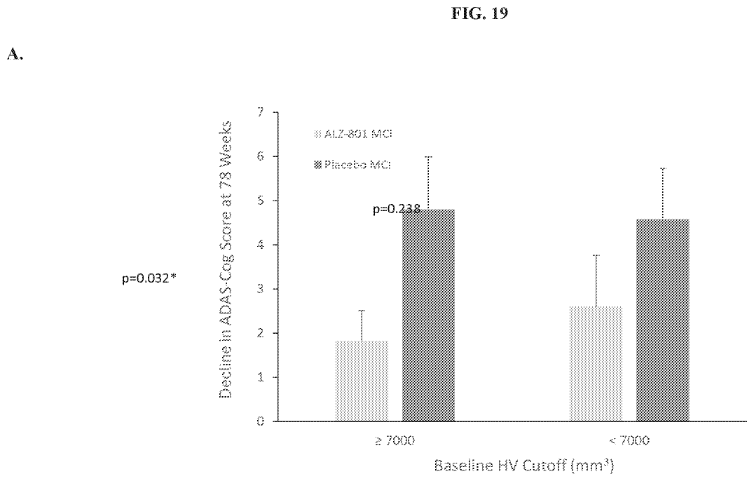

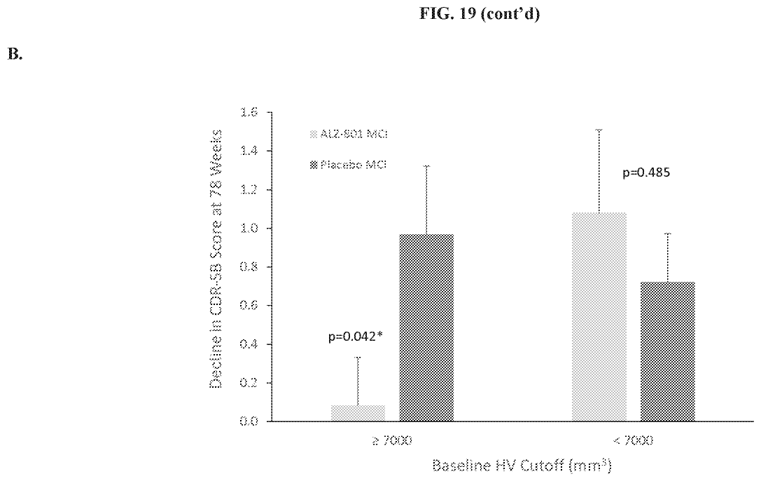

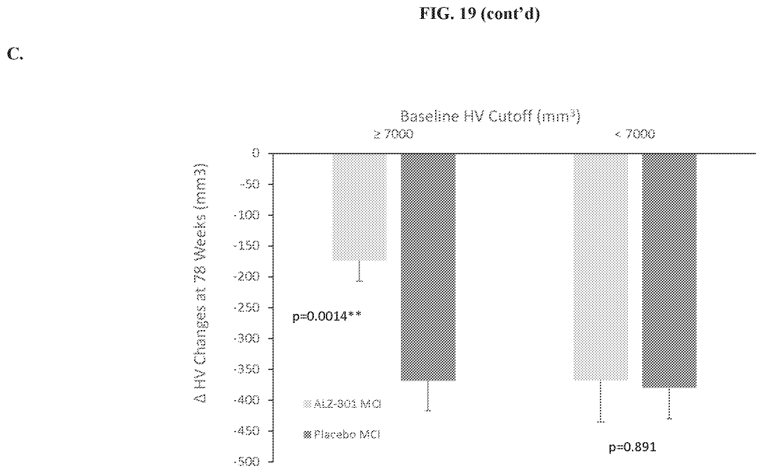

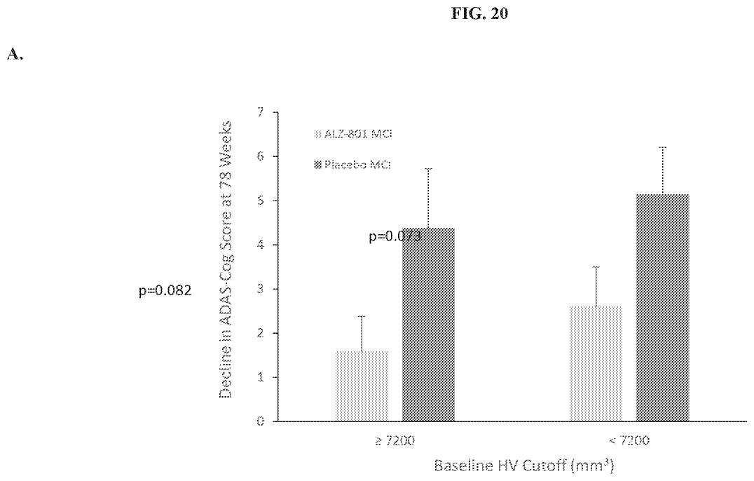

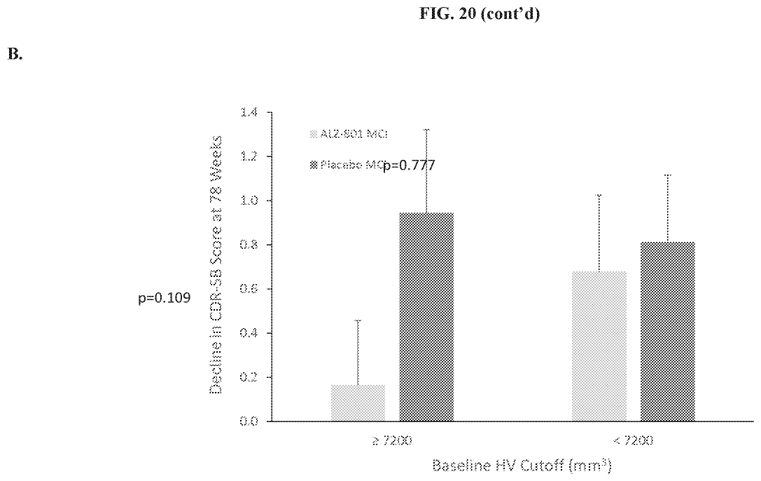

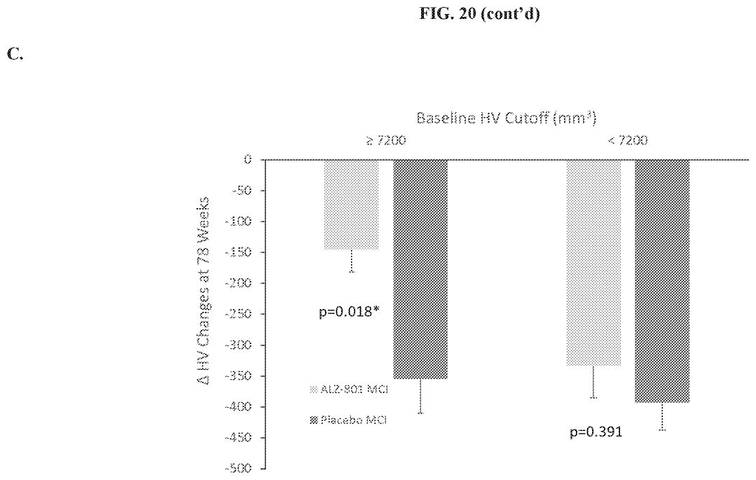

In order to further investigate the effect of baseline HV on subjects' response to valiltramiprosate treatment, we divided the MCI subjects into two subgroups (equal to or above an HV baseline vs below an HV baseline) using three different HV baseline cutoffs (6600 mm³, 7000 mm³ and 7200 mm³). The effect of 78 weeks of valiltramiprosate treatment on three different parameters—ADAS-Cog scores, CDR-SB scores, and HV—was compared to placebo treatment for each subgroup. The results showed that for each HV baseline cutoff analyzed, the group of valiltramiprosate-treated MCI subjects at or above the cutoff demonstrated a statistically significant improvement in at least one of the three parameters as compared to the corresponding placebo control (FIGS. 18-20). In contrast, each of the groups of valiltramiprosate-treated MCI subjects below the HV baseline cutoffs failed to demonstrate a statistically significant improvement in any of those three parameters as compared to the corresponding placebo control.

### Example 2. Phase 2 Human Clinical Trial Long-Term Extension Results

A Phase 2, single-arm, study enrolled 53 APOE3/4 and 31 APOE4/4 Early AD subjects (MMSE≥22, CDR-G=0.5, 1) with positive amyloid-PET or CSF AD biomarkers. Subjects orally administered 265 mg valiltramiprosate BID for the 104 week initial trial and for an additional 104 week long-term extension. Seventy (70) subjects completed the initial 104-week trial, 65 subjects completed an additional 52 weeks of the long-term extension, and 51 subjects completed an additional 104 weeks of the long-term extension. Subjects were administered the MMSE, RAVLT, CDR-SB tests before being dosed with valiltramiprosate, as well as at various times during the trial (Weeks 13, 26, 52, 78, 104, 130, 156, 182, and 208).

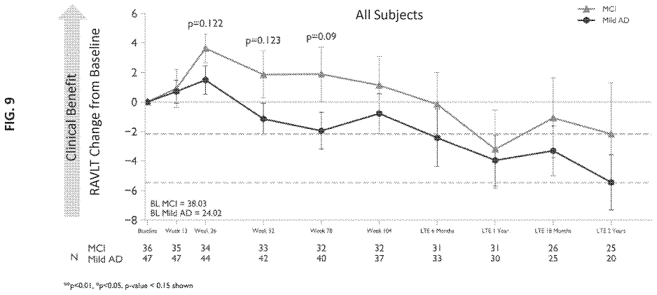

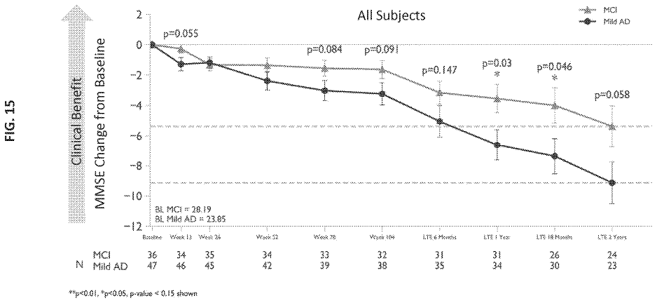

A subgroup analysis of the results of this 4-year clinical trial by separating subjects into those who began the trial with Mild AD (baseline MMSE 22-26) and those who began the trial with MCI (baseline MMSE 27-30) was completed. Surprisingly, subjects with MCI demonstrate greater clinical benefit than those with Mild AD. As can be seen from FIG. 9, subjects with MCI demonstrated an average of no change from baseline in the RAVLT over the 4 year trial, while those with Mild AD showed an average of an approximate 9-point drop in RAVLT scores over the same period. Similarly, FIG. 12 shows that the MCI subjects had an approximately 3-point drop from baseline in CDR-SB scores, while Mild AD subjects had a 6-point drop. The same trend can also be seen in MMSE scores. FIG. 15 shows that MCI subjects had an average drop of 5.5 points in MMSE score after 4 years, while the MMSE score for Mild AD subjects dropped about 9 points. The table below shows the percentage of subjects in each group that demonstrated stable MMSE at 104 weeks, 156 weeks and 208 weeks of treatment. Stable MMSE is defined as ≤4-point drop in MMSE compared to baseline.

| | Stable MMSE | | | |
|---|---|---|---|---|
| Subgroup | Week 104 | Week 156 | Week 208 | |
| MCI (MMSE 27-30) | 26/31 (84%) | 21/30 (70%) | 14/24 | (58%) |
| Mild AD (MMSE 22-26) | 26/39 (67%) | 15/35 (43%) | 8/27 | (30%) |

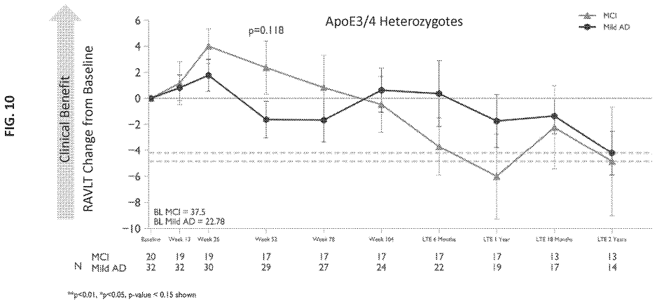

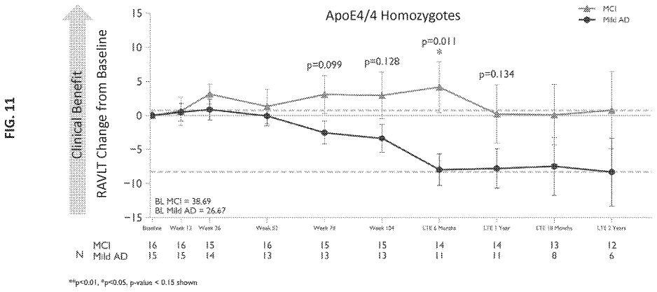

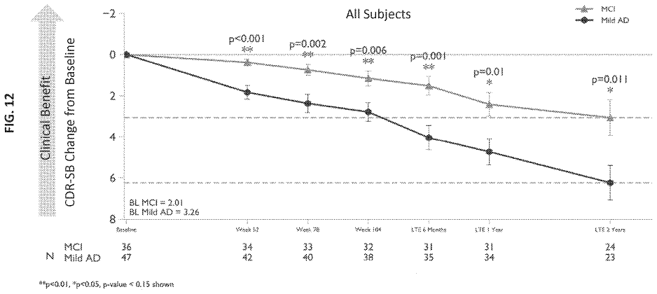

We next performed a further subgroup analysis based on both severity of disease (MCI vs Mild AD) and ApoE4 genotype (ApoE4/4 homozygotes or ApoE3/4 heterozygotes). Interestingly, for ApoE3/4 heterozygotes, RAVLT score changes from baseline for MCI and Mild AD subjects varied greatly over time and after 4 years, there seemed to be little difference between those two subgroups (FIG. 10). For ApoE4/4 homozygotes there was an average of almost a 9 point average decline from baseline for Mild AD subjects (i.e. subjects with more advanced disease severity), compared to no change for MCI subjects (FIG. 11).

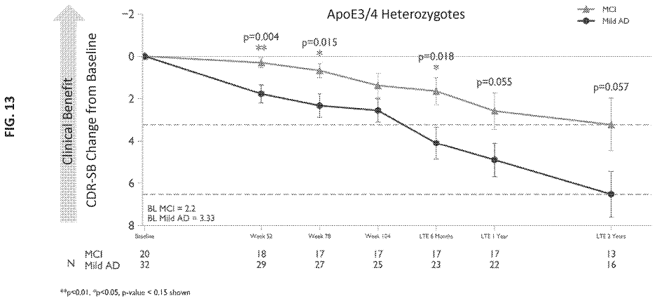

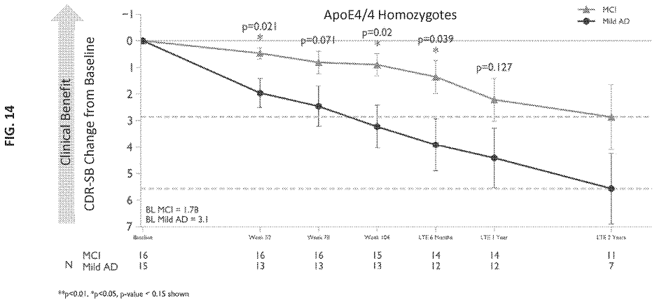

For CDR-SB scores (CDR is a clinical outcome that incorporates both cognition and functional assessments), in both the ApoE3/4 heterozygote (FIG. 13) and ApoE4/4 homozygote (FIG. 14) subgroups, the MCI group demonstrated superiority over the Mild AD group in terms of reduced decline from baseline. The ApoE3/4 heterozygote MCI group dropped an average of about 3.5 points from baseline after 4 years while the MCI group dropped about 6.5 point. In the ApoE4/4 homozygote subjects, the MCI subgroup dropped an average of about 3 points from baseline, which the Mild AD group dropped about 5.5 points.

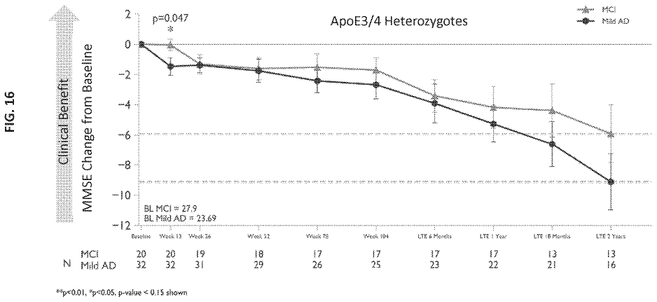

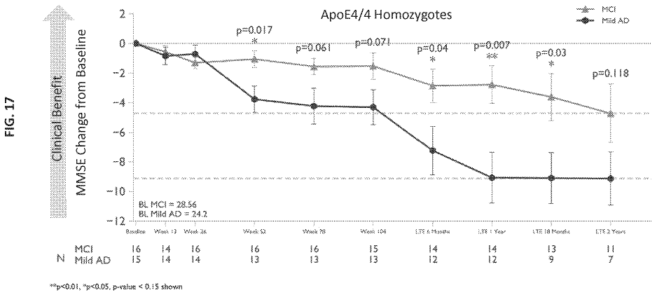

The ApoE3/4 heterozygote and ApoE4/4 homozygote MCI subgroups also both showed superiority to the corresponding Mild AD subgroups in terms on MMSE score decline. The ApoE3/4 heterozygote MCI subgroup showed an average of about a 6 point drop in MMSE score after 4 years, while the corresponding Mild AD subgroup showed an average 9.5 point drop (FIG. 16). The ApoE4/4 homozygote MCI subgroup showed an average of about a 4.5 point drop in MMSE score after 4 years, while the corresponding Mild AD subgroup showed an average 9.25 point drop (FIG. 17).

For each of the graphs in FIGS. 9-17, the change from baseline is measured on a subject-by-subject basis, using each particular subject's baseline score. Those changes are then averaged and plotted in these graphs.

The table below shows the percentage of subjects in each subgroup that demonstrated stable MMSE at 104 weeks, 156 weeks and 208 weeks of treatment. Stable MMSE is defined as ≤4-point drop in MMSE compared to baseline.

| Disease Severity | ApoE | Stable MMSE | | | | | |
|---|---|---|---|---|---|---|---|
| Subgroup | Genotype | Week 104 | | Week 156 | | Week 208 | |
| MCI (MMSE 27-30) | ApoE3/4 | 14/17 | (82%) | 12/17 | (71%) | 6/13 | (46%) |
| Mild AD (MMSE 22-26) | ApoE3/4 | 17/25 | (68%) | 11/22 | (50%) | 7/18 | (39%) |
| MCI (MMSE 27-30) | ApoE4/4 | 12/14 | (86%) | 9/13 | (69%) | 8/11 | (73%) |
| Mild AD (MMSE 22-26) | ApoE4/4 | 9/14 | (64%) | 4/13 | (31%) | 1/9 | (11%) |

At each time point and for each genotype, the MCI subgroup had a higher percentage of subjects who had stable MMSE as compared to the corresponding Mild AD subgroup.

In addition, the correlation between hippocampal volume changes and MMSE scores during the long-term extension of the phase 2 human clinical trial was analyzed. The results show a statistically significant and strong correlation between change in hippocampal volume and change in MMSE scores from baseline at the start of the trial after 104 weeks, after 156 weeks, and after 208 weeks on valiltramiprosate. The correlations all showed a p value of <0.01, except for the MCI population at baseline. The correlations were slightly higher in the MCI subpopulation as compared to the overall trial population (mix of MCI and Mild AD). This further demonstrates that preservation of hippocampal volume plays an important role in preserving cognitive function, and thus a predictor of clinical cognitive benefit in AD.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) that may be cited throughout this application are hereby expressly incorporated herein in their entireties by reference. Unless otherwise defined, all technical and scientific terms used herein are accorded the meaning commonly known to one with ordinary skill in the art.

1. A method of selecting and treating a human subject to prevent, delay or slow the progression of MCI to Alzheimer's disease comprising the steps of:
a. selecting a subject only if the subject: (i) is ApoE4-positive, (ii) is less than 80 years old, (iii) is diagnosed with mild cognitive impairment prior to treatment, and (iv) has a base hippocampal volume of at least 6600 mm³; and
b. treating the selected subject with an effective amount of valiltramiprosate.

2. The method of claim 1, wherein the effective amount of valiltramiprosate is 265 mg BID.

3. The method of claim 1, wherein the subject is selected only if the subject has a base hippocampal volume of at least 7000 mm³, at least 7200 mm³, at least 7250 mm³, at least 7500 mm³, at least 7750 mm³, at least 8000 mm³, at least 8250 mm³, at least 8500 mm³, at least 8750 mm³, or at least 9000 mm³.

4. The method of claim 3, wherein the subject is selected only if the subject has a base hippocampal volume of at least 7000 mm³ or at least 7200 mm³.

5. The method of claim 1, wherein the subject is selected only if the subject is less than 70 years old, less than 69 years old, less than 68 years old, less than 67 years old, less than 66 years old, less than 65 years old, less than 64 years old, or less than 63 years old upon the initial administration of valiltramiprosate.

6. The method of claim 1, wherein the subject is selected only if the subject is also ApoE4 homozygous.

7. The method of claim 1, wherein the subject is diagnosed with mild cognitive impairment on the basis of one or more of the following:
a. one or more different cognitive test thresholds;
b. one or more cognitive test thresholds coupled with one or more brain microstructure thresholds as determined by Diffusion Tensor Imaging (DTI);
c. one or more cognitive test thresholds coupled with one or more plasma or cerebral spinal fluid biomarker thresholds; or
d. one or more cognitive test thresholds coupled with brain amyloid and/or Tau thresholds as determined by positron emission tomography (PET).

8. The method of claim 6, wherein the subject is diagnosed with mild cognitive impairment on the basis of a MMSE score of 26-30, a CDR-G score of 0.5, a RBANS-DM index of ≤105, and positivity for plasma pTau217.

9. The method of claim 6, wherein the subject is diagnosed with mild cognitive impairment on the basis of a MMSE score of 26-30, a CDR-G score of 0.5, a RBANS-DM index of ≤95, and positivity for plasma pTau217.

10. The method of claim 6, wherein the subject is diagnosed with mild cognitive impairment on the basis of a MMSE score of 26-30, a CDR-G score of 0.5, a RBANS-DM index of ≤95, and positivity for plasma pTau217/Aβ42 ratio.

11. The method of claim 6, wherein the subject is diagnosed with mild cognitive impairment on the basis of a MMSE score of 26-30, a CDR-G score of 0.5, a RBANS-DM index of ≤105, and positivity for plasma pTau217/Aβ42 ratio.

12. The method of claim 1, wherein the subject is only selected if the subject is diagnosed with mild cognitive impairment within six months prior to treatment.

13. The method of claim 11, wherein the subject is only selected if the subject is diagnosed with mild cognitive impairment within ninety days prior to treatment.

14. The method of claim 11, wherein the subject is only selected if the subject is diagnosed with mild cognitive impairment within sixty days prior to treatment.

15. The method of claim 1, wherein the subject is treated with an effective amount of tramiprosate for at least 52 weeks, at least 78 weeks, at least 104 weeks, at least 130 weeks at least 156 weeks, at least 182 weeks, or at least 208 weeks.
